(19) United States
(12) Patent Application Publication
(10) Pub. No.: US 2024/0306980 A1
(43) Pub. Date: Sep. 19, 2024

(54) PATIENT-SIDE, REAL-TIME SEPSIS MONITORING

(71) Applicants: Healcisio, Inc., San Diego, CA (US); The Regents of the University of California, Oakland, CA (US)

(72) Inventors: Hooman SEDGHAMIZ, San Diego, CA (US); Shamim NEMATI, San Diego, CA (US); Hayden H. POUR, San Diego, CA (US)

(21) Appl. No.: 18/608,822

(22) Filed: Mar. 18, 2024

Related U.S. Application Data
(60) Provisional application No. 63/490,896, filed on Mar. 17, 2023.

(51) Int. Cl. A61B 5/00; A61B 5/01; G16H 10/60; G16H 50/30

(52) U.S. Cl. CPC A61B 5/412; A61B 5/002; A61B 5/0022; A61B 5/01; A61B 5/4842; A61B 5/7405; A61B 5/742; G16H 10/60; G16H 50/30; A61B 2560/0406; A61B 2560/0462; A61B 2562/0257

(57) ABSTRACT

Patient personal output apparatuses and methods of making and using them. A patient personal output apparatus may be configured as tableside and/or bedside digital companion (e.g., ‘pet’) that, once paired with a patient health data, may display an indicator of the patient's risk of a decompensation event, such as sepsis.

### CLAIM OF PRIORITY

This patent application claims priority to U.S. Provisional Patent Application No. 63/490,896, titled “PATIENT-SIDE, REAL-TIME SEPSIS MONITORING,” filed on Mar. 17, 2023, and herein incorporated by reference in its entirety.

### BACKGROUND

Sepsis is a leading cause of morbidity and mortality, affecting millions of patients every year. Early detection of sepsis is vital because it allows for the timely administration of life-saving treatments. The majority of cases of sepsis are community on-set, and the rate of patients readmitted due to sepsis is substantial. Therefore, continuous monitoring of patients at risk for sepsis across the continuum of care could potentially allow the early detection of sepsis and improve population care management of this vulnerable patient population.

While predictive models for early prediction of sepsis using electronic health records (EHR) have been proposed, effective implementation and workflow integration of such tools at the bedside has proven elusive. Further, there are no existing systems for monitoring these patients beyond hospital settings. Some of the barriers to effective bedside implementation of such systems include the closed nature of electronic health records, issues surrounding patient privacy, HIPAA regulations, and ownership of healthcare data, and the difficulty of integration of multimodal data sources from EHR and other healthcare-related medical devices (e.g., the “internet of medical things” or IoMT).

Described herein are methods and apparatuses (e.g., systems, devices, etc. including software, hardware and firmware) that may address these issues. In particular, the methods and apparatuses described herein may improve continuous patient monitoring and facilitate precision medicine, and may provide an integrated health tracking system that can be dynamic, ubiquitous, less expensive (low cost), and more independent than other proposed solutions.

### SUMMARY OF THE DISCLOSURE

Described herein are patient personal output apparatuses, e.g., systems and devices, including computer software, hardware and/or firmware, etc., methods of making patient personal output apparatuses, and using patient personal output apparatuses. These patient personal output apparatuses may be configured as tableside or bedside digital companions (‘companion pets’) that, once paired with a patient, may determine, in real time, a risk of a decompensation event, such as sepsis, kidney failure, heart failure, respiratory failure, 30 days hospital readmission, etc.

For example, a patient personal output apparatus may include: an outer body configured to be positioned near a patient; one or more outputs coupled to the outer body; a control circuitry within the outer body, the control circuitry comprising: a communications module configured to communicate with one or more of: the patient's electronic health record, or a monitoring device monitoring the patient to receive patient data; a decompensation scoring module comprising a trained neural network configure to output a decompensation score based on the patient data; and an output module configured to output a representation of the decompensation score from the one or more outputs.

In any of these examples the patient personal output apparatus, and in particular the outer body of the patient personal output apparatus, may be configured as an anthropomorphized animal shape. In some examples the outer body is configured as an anthropomorphized animal shape comprising a cartoon animal or mythical creature. Examples of anthropomorphized animal shapes include mammalian shapes (dogs, monkeys, cats, cows, horses, etc.), fish, birds, insects, arthropods (crabs, lobster), etc. The anthropomorphized animal shape may be mythical animals (unicorns, griffons, mermaids, etc.).

The apparatus may include any output. For example, the one or more outputs may comprise one or more LEDs. The one or more outputs may comprise a screen, display, projector, or speaker. In some examples the body of the patient personal output apparatus is configured to be transparent or translucent and all or a region of the patient personal output apparatus may be configured to be illuminated (e.g., glow, emit light, etc.) that is related to the decompensation score (e.g., risk).

The control circuitry may include any appropriate circuitry element configured to perform the functions described herein. For example, the control circuitry may include a memory, e.g., for storing data from the patient's electronic health record, or a monitoring device monitoring the patient. In any of these apparatuses the patient personal output apparatus may include one or more sensors (e.g., non-contract sensors). For example, the control circuitry may further comprise one or more non-contact sensors. In some examples, the one or more non-contact sensors comprises a camera, a LADAR subsystem, or infrared thermography subsystem.

In some examples the control circuitry may further comprise a patient verification module configured to verify the identity of the patient. The decompensation scoring module may comprise a trained neural network configure to output a decompensation score representing risk of sepsis based on the patient data.

For example, described herein are patient personal output apparatus comprising: an outer body configured to be positioned near a patient, wherein the outer body is configured as an anthropomorphized animal shape; one or more outputs coupled to the outer body; a control circuitry within the outer body, the control circuitry comprising: a patient verification module configured to verify the identity of the patient; a communications module configured to communicate with one or more of: the patient's electronic health record, or a monitoring device monitoring the patient to receive patient data; a decompensation scoring module comprising a trained neural network configure to output a decompensation score representing risk of sepsis based on the patient data; and an output module configured to output a representation of the decompensation score from the one or more outputs by modulating a light emitted by the one or more outputs.

Also described herein are methods of using these apparatuses. For example, A method of using a patient personal output apparatus to determine and/or display a risk of a decompensation event may include: receiving data specific to a patient from one or more of: a patient's electronic health record and/or a monitoring device associated with the patient, wherein the data is received by a control circuitry of a patient personal output apparatus; determining, from a decompensation module of the patient personal output apparatus, a decompensation score based on the received data; and outputting a visual indicator of the decompensation score from the patient personal output apparatus, wherein the patient personal output apparatus is positioned proximate to the patient.

Any of these methods may include associating the patient with the patient personal output apparatus. The patient may be associated by confirming and/or verifying that the patient identity of a patient is associated with a patient personal output apparatus.

In any of these methods, determining the decompensation score based on the received data may comprise determining a risk of one or more of: sepsis, kidney failure, heart failure, or respiratory failure. For example, determining the decompensation score based on the received data may comprise determining a risk of sepsis. In some examples determining the decompensation score based on the received data comprises determining the decompensation score in real time and/or in an ongoing manner. Determining a risks score may require the device to keep track of the patient baseline data and maintain a model of normal patient data, or to establish a notion of normality that combines population level data with patient-level individualized data. For instance, one may use a probabilistic (or variational) auto-encoder, or any type of generative probabilistic model, to learn a model of data from a population of normal patients or from the patient's baseline data, and use the model to detect deviations from this normality (for instance, by calculating the auto-encoder reconstruction error or applying extreme-value detection methods to the data joint distribution).

Outputting the visual indicator of the decompensation score from the patient personal output apparatus may comprise modulating a light emitted by the patient personal output apparatus while the patient personal output apparatus is positioned proximate to the patient.

In any of these examples outputting the visual indicator of the decompensation score comprises changing the appearance of a region of the patient personal output apparatus corresponding to a region of the body related to the decompensation score.

For example, a method may include: associating the patient with a patient personal output apparatus that is positioned proximate to the patient, wherein the patient personal output apparatus comprises an anthropomorphized animal shape; receiving data specific to a patient from one or more of: a patient's electronic health record and/or a monitoring device associated with the patient, wherein the data is received by a control circuitry of a patient personal output apparatus; determining, from a decompensation module of the patient personal output apparatus, a decompensation score indicating a risk of sepsis in real time in an ongoing manner, and based on the received data; and outputting a visual indicator of the decompensation score from the patient personal output apparatus by modulating a light emitted by the patient personal output apparatus while the patient personal output apparatus is positioned proximate to the patient.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates one example of a patient personal output apparatus.

FIG. 2 illustrates one example of a patient personal output apparatuses, with control circuitry and analytics included inside the apparatuses.

FIG. 3 schematically illustrates an example of an architecture for a control circuitry for a patient personal output apparatus as described herein.

FIG. 4 schematically illustrates an example of networked apparatuses for patient location tracking via triangulation.

### DETAILED DESCRIPTION

Described herein are patient personal output apparatuses (e.g., systems and devices, including computer software, hardware and/or firmware) and methods of making and using patient personal output apparatuses. These patient personal output apparatuses may be configured as tableside or bedside digital companions (‘pets’) that, once paired with a patient, may securely access and/or receive data from the patient's medical or health record (or records), one or more patient monitors (including third party monitors or monitoring systems) and/or digital health devices (e.g., wearables, such as smartwatches, etc.). The patient personal output apparatuses described herein may reliably and efficiently verify patient identity, and may provide a visible and/or or audible output to the patient and local caregivers (e.g., doctors, nurses, etc.) of decompensation risks, in particular, risk of sepsis, respiratory failure, kidney failure, etc. Risk may be indicated by a simplified output interface so that the patient risk may be readily identified.

Decompensation risks may be determined using one or more trained neural networks that are part of the patient personal output apparatus. The patient personal output apparatus may receive patient information, including ongoing continuous monitoring information, that may be used as input to the trained neural network or an anomaly detection algorithm.

For example, the patient personal output apparatuses described herein may incorporate a trained neural network for the detection of Sepsis. A number of different approaches for the determination and identification of Sepsis risk have been proposed. For example, see Shashikumar et al. (“Artificial intelligence sepsis prediction algorithm learns to say ‘I don't know’. NPJ Digit. Med. 4, 134 (2021). doi: 10.1038/s41746-021-00504-6), and Nemati et al. (Nemati S, Holder A, Razmi F, Stanley M D, Clifford G D, Buchman T G. An interpretable machine learning model for accurate prediction of sepsis in the ICU. Critical care medicine. 2018 April; 46(4):547. doi: 10.1097/CCM.0000000000002936).

The patient personal output apparatuses described herein may act as local identification and output units that associated with the patient, and may remain in proximity to the patient (e.g., within the same room, such as bedside, tableside, etc.) with the patient, and may permit the rapid and effective display of risk such as sepsis risk (or other decompensation risks).

In some examples the patient personal output apparatuses may be configured as a compact unit that includes control and analysis circuitry within a local housing that is configured for outputting a current risk level for one or more decompensation risks. The local housing may be configured as a “pet” or companion having an animal (real or fanciful) shape. The local housing of the patient personal output apparatuses may be configured as a homuncular representation of a human or animal (or anthropomorphized animal). These shapes may be referred to as “cartoon” shapes and styles (e.g., cartoon dogs, cats, birds, cows, or other animals, plants, insects, etc.). The patient personal output apparatus may be configured so that the output is represented as a change in appearance of the local housing. For example, the patient personal output apparatuses may be configured to change color and/or intensity to indicate risk of one or more decompensations. In some examples the local housing may be configured as a translucent or transparent body that is illuminated (e.g., from within) by one or more lights (e.g., LEDs) in a color and/or intensity to indicate a level or state of decompensation risk.

The patient personal output apparatus may be configured as a soft (e.g., plush) outer body, such as a stuffed animal, and/or may be at least partially rigid. The patient personal output apparatus may be configured to sit upright, e.g., on a smooth or uneven surface, or may be configured for attachment to surface (table, floor, wall, etc.). In some example the patient personal output apparatus may be configured to attach to a bed, chair (e.g., wheelchair), etc.

In one example a patient personal output apparatus may be configured to use one or more deep learning model for the early detection (e.g. prediction) of a decompensation such as sepsis. In general the decompensation detection may be based on inputs from the patient health record (e.g., electronic health record or EHR), and/or inputs from one or more patient-associated monitors (e.g., wearable fitness watches or bio-patches). Patent data, including patient health data may be used by the one or more deep learning models (e.g., trained neural networks) to provide an output of the likelihood of a decompensation event such as sepsis. The likelihood may be expressed as a numeric value or score and may be adapted to work in the patient personal output apparatus as an ongoing output. For example the patient personal output apparatus may be configured to provide a user-friendly presentation of the likelihood of a decompensation event such as sepsis, which may address several limitations of current systems. The patient personal output apparatus (or “pet”), which may include control circuitry such as one or more processors (implemented in one prototype on a raspberry pi) and outputs (e.g., display, projector, LED lights, etc.), may be placed near the patient, such as next to (or in) the patient bed in an in-patient (e.g., hospital) settings or other settings (e.g., home, hospice, etc.) where patients are monitored for their physiological status.

The patient personal output apparatus may collect patient information (clinical data) from the patient's electronic health records (EHR). For example, the patient personal output apparatus may use Fast Healthcare Interoperability Resources (FHIR) Application Programming Interface (API) calls, as well as other internet of medical things (IoMT) devices, and may process and generate features for prediction by the trained deep learning model to continuously produce risk scores, e.g., for sepsis and/or for a probability of hospital readmission. In some examples, the LED color and intensity of the patient personal output apparatus may be adjusted based on the predicted risk scores, which may provide an immediate visual notification to the caregivers of an impending deterioration event.

The patient personal output apparatus can operate within a hospital network, and in particular, the patient personal output apparatus may establish its own secure patient-specific network, and thus can address the issues of data privacy and ownership. These apparatuses may also be configured to establish a stand-alone and user-friendly user interface, and can integrate data from multiple sources, and perform all computations on the device (e.g., ‘edge computing’).

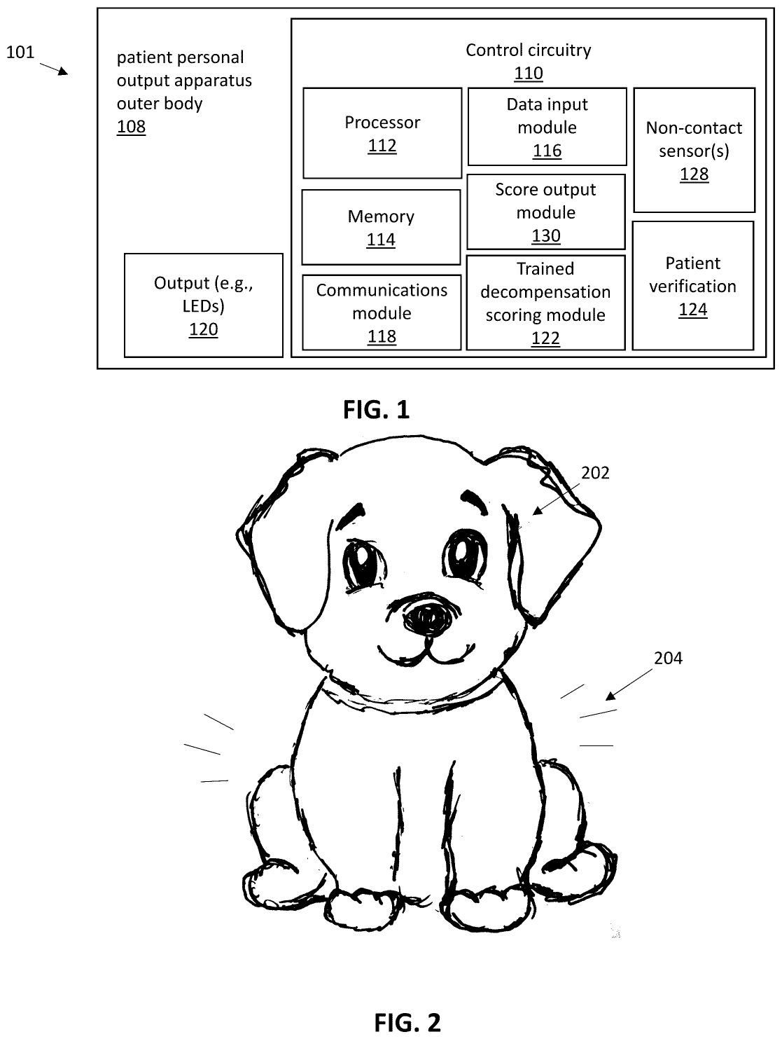

For example, FIG. 1 schematically illustrates one example of a patient personal output apparatus 101 including control circuitry 110 which may include or may couple with one or more processors 112, memory 114, a data input module 116, a communications module 118 (which may be part of or connected to the data input module in some examples), and one or more trained decompensation scoring modules 122. The apparatus may also include one or more outputs, including in some examples an LED or set of LEDs 120 for outputting the results of the scoring module 122. These components may be enclosed or housed within an outer body, as described above. The outer body may be formed in the shape of a ‘pet’ (e.g., a cartoon animal, etc.). The LEDs may illuminate all or a portion of the outer body and a characteristic of the output may encode the score (risk factor) from the trained decompensation scoring module. The output may encode the score for presentation by the output. Other outputs may include a visual output (e.g., to a screen, display, projector, etc.), audible output (e.g., tone, etc.), text, etc. including combinations of any of these. The output of the decompensation scoring module may be a score that may be processed for the output 120 by a score output module 120.

The modules forming part of the control circuitry may include one or more engines and datastores. As used herein, a module or engine may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine or module and a second engine or module can have one or more dedicated processors, or a first engine or module and a second engine or module can share one or more processors with one another or other engines. Depending upon implementation-specific or other considerations, an engine can be centralized, or its functionality distributed. An engine or module can include hardware, firmware, or software embodied in a computer-readable medium for execution by the processor. The processor transforms data into new data using implemented data structures and methods, such as is described with reference to the figures herein.

The modules and sub-systems described herein can be implemented locally. In some examples a portion of these module or sub-systems can be cloud-based. As used herein, a cloud-based module or sub-system may run applications and/or functionalities using a cloud-based computing system. All or portions of the applications and/or functionalities can be distributed across multiple computing devices, and need not be restricted to only one computing device. In some embodiments, the cloud-based module or sub-system can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores described herein can be local or, in some examples, cloud-based datastores. A cloud-based datastore is a datastore that is compatible with cloud-based computing systems and engines.

Any of these patient personal output apparatuses described herein may also optionally include a patient verification module 124, that may verify the patient's identity and/or may pair the patient to the apparatus. In some examples, patient verification may be based on, or may include, patient proximity. For example, the apparatus may verify and confirm the patient's identity by voice recognition, by matching the patient to one or more wearable (e.g., identification tags), etc. The apparatuses described herein may also optionally include integrated sensors, such as, e.g., non-contact sensors 128.

In some examples the proposed patient personal output apparatus may address the global burden of sepsis by providing methods and apparatuses for early detection and prevention of sepsis that covers the continuum of care, from hospital bedside to nursing homes to patients' homes. The patient personal output apparatus may leverage the power of the Internet of Medical Things (IoMT) and deep learning algorithms to accurately predict the onset of a decompensation (e.g., such as, but not limited to, sepsis) in patients.

For example, these apparatuses may access a patient's Electronic Health Records (EHRs) and FHIR APIs, as well as other IoMT APIs. The patient personal output apparatus may collect patient data in real, or near-real time (e.g., at an hourly resolution) and may use this information to calculate a decompensation score (e.g., a Sepsis Score), which may then displayed through one or more output, such as calibrated LED lights. For example, FIG. 2 illustrates an example of a patient personal output apparatus configured to have an outer body that is shaped like a cartoon dog 202. The outer body is at least partially translucent, and may be configured so that one or more LEDs may be controlled by the control circuitry so that the color and intensity of the lights 204 are adjusted based on the predicted risk score, providing bedside caregivers and/or patients with a visual indicator of impending severe sepsis events. In some examples the LED light sources can be positioned relative to the patient personal output apparatus's human-analogous anatomical locations to highlight potential sources of infection or specific areas of organ damage and impending physiological deterioration (e.g., head, torso, arms, legs, etc.).

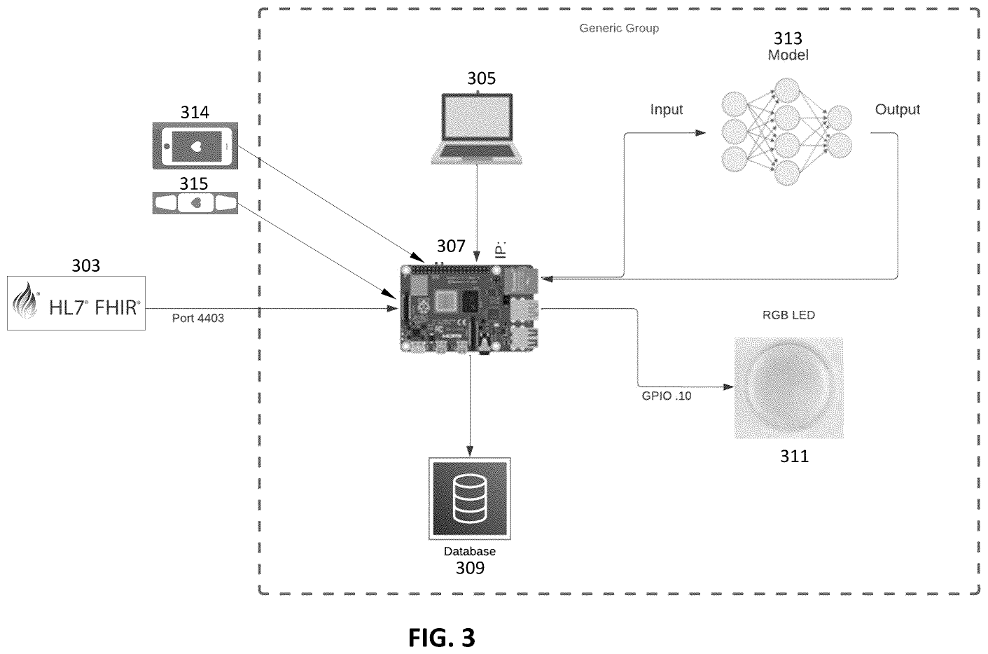

FIG. 3 schematically illustrates one example of an architecture for a control circuitry configuration that may be used or modified as described herein for a patient personal output apparatus. In FIG. 3, the control circuitry includes one or more processors 307 that may access a local (or in some cases, remote) database or data structure 309. The control circuitry may also include and/or access a trained neural network 313 (“Model”) that may receive inputs extracted from the patient's electronic health record (EHR). For example, the apparatus may include a remote connection to the processor (e.g., a Raspberry Pi) having an IP addresses, through a protected computer 305 to synchronize with a patient Encounter. In FIG. 3, the “Epic on FHIR” 303 is an API to Epic EHR data for collecting patient data. The apparatus may be configured to use SQLite for small, fast, and self-contained databases. In one example the trained neural network is ‘Tensorflow Lite’ which is configured to predict Sepsis scores. The model output (predicted score) from the trained neural network may be used to update the output, e.g., LEDs 311 on or in the body of the patient personal output apparatus to notify bedside caregiver.

The use of a patient personal output apparatus that is local to the patient and provide secure access to the patient data, as well as immediate indication of the likelihood of one or more decompensation events is a significant improvement over existing EHR-based alerting systems, and may provide a simpler and more effective way of notifying clinicians of a potential sepsis episode, without the need for disruptive EHR-based pop-ups and loud beeping sounds/audio alerts. The use of integrated trained neural networks (e.g., trained by deep learning algorithms) and IoMT technology may provide a low-cost, dynamic, and ubiquitous solution to the problem of monitoring and/or preventing decompensation events such as sepsis. The methods and apparatuses described herein may providing an early warning system for decompensation events that may significantly reduce morbidity and mortality, particularly those associated with sepsis, improving patient outcomes and quality of life.

Any of the apparatuses and methods described herein may providing an innovative solution for early detection and prevention of decompensation (e.g., sepsis) that may cover the continuum of care, from hospital bedside to nursing homes to patients' homes. The patient personal output apparatus may leverage the power of the Internet of Medical Things (IoMT) and deep learning algorithms to accurately predict the onset of a decompensation such as sepsis in patients. As mentioned, the patient personal output apparatus may extract data from the patient's Electronic Health Records (EHRs) using FHIR APIs, as well as other IoMT APIs. In some examples the patient personal output apparatus may be configured to collect patient data at an hourly resolution and uses this information to calculate a score (e.g., a sepsis score), which may then be output, e.g., displayed, through one or more outputs such as through calibrated LED lights. A data output module may determine the output, for example, such as the color and/or intensity of the lights and may adjust them based on the predicted risk score, providing bedside caregivers with a visual indicator of impending severe sepsis events. As mentioned, in some examples the output can be placed on patient personal output apparatus's anatomical locations to highlight potential sources of infection or specific areas of organ damage and impending physiological deterioration.

In general the patient personal output apparatus may be used in an inpatient and/or outpatient setting, including in a home setting. For example the apparatus may be used even where electronic medical records are not immediately available, e.g., the use of these patient personal output apparatuses is not limited to gathering patient data from EHRs and FHIR APIs. In some examples the patient personal output apparatus can be configured to connect to one or more wearable health monitoring devices, such as Apple™ and Samsung™ watches, Fitbits™, etc. This may allow the apparatus to detect and/or prevent decompensation (e.g., sepsis) not only in the community, not limited to inpatient settings. As an example, these apparatuses may combine continuous accelerometry data (e.g., from a Fitbit™ or other patient monitoring device) and/or may use (and combined with) information from the patient's medical record to enhance the prediction of sepsis readmission. The use of wearable health monitoring devices may allow for continuous monitoring and early detection, even outside of the hospital setting, improving the chances of successful treatment and recovery. The integration of wearable technology into the patient personal output apparatus expands the reach and impact of the solution, further improving patient outcomes.

### Patient Identification

In order to ensure the accuracy of the patient personal output apparatus, multiple methods of patient identification and authentication may be integrated as part of the patient personal output apparatus. In some examples the apparatus is equipped with voice verification, allowing patients to be identified by their unique vocal characteristics. Alternatively or additionally, the apparatus may utilize location monitoring (GPS) to confirm the patient's location, ensuring that the correct patient is being monitored. In some examples, the apparatus may include a proximity sensor, e.g., using blue-tooth low energy (BLE) technology or others sensing modality (e.g., ultrawideband, RF, etc.) that allows it to detect nearby patient identifiers. This combination of voice verification, location monitoring, and/or wearable (e.g., tag or wristband) identification may provide a robust and secure system for patient identification and authentication, ensuring that the correct patient data is being used and analyzed by the apparatus.

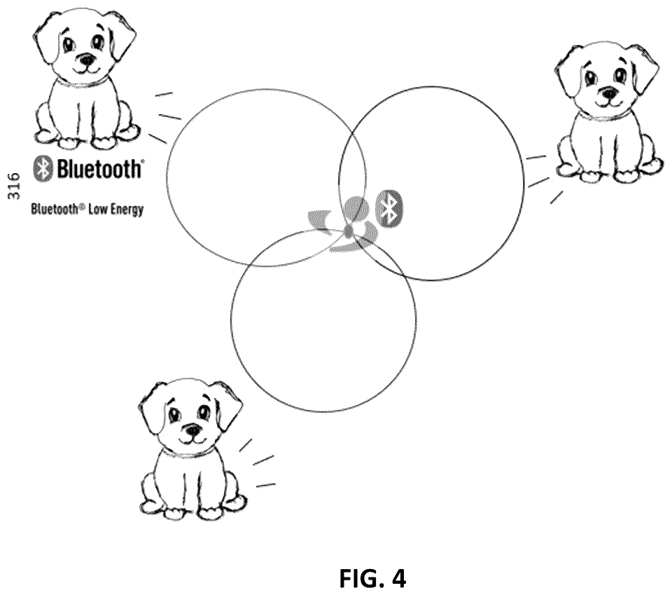

In some examples the patient personal output apparatus may be used to track movements of patient's and/or health care providers. For example, any of these apparatuses may track the movements of patients or caregivers between different regions of a hospital. For examples, patient personal output apparatuses located in different hospital rooms can track the movements of any nearby patient or caregiver with a BLE-transmitting device (or other tracking modality), via triangulation (see FIG. 4). The patient personal output apparatus may use this tracking information, as well as information about the patient-specific probability of infection (independent of the organism type) to track infection transmission throughout the hospital system. The use of bedside devices such as the patient personal output apparatuses described herein may be combined with proximity sensors to enhance infection control in hospitals and reduce the risk of transmission to patients, staff, and visitors.

In some examples the apparatuses described herein may use one or more algorithms to rank tracking data. For example, these apparatuses may use an algorithm similar to PageRank, (typically used by search engines to rank web pages) to estimate high-risk areas in hospitals, i.e., areas with the highest level of infection and frequent traffic to other locations. These algorithms can be applied to the data collected by bedside devices and proximity sensors, identifying areas where a virus is more likely to be present based on factors such as proximity to infected individuals, frequency of use, and ventilation. By analyzing this data, the patient personal output apparatus, in combination with other nearby patient personal output apparatuses, can rank areas in the hospital according to their risk level, allowing staff to focus their infection control efforts where they are needed most. This approach can help hospitals to optimize their infection control strategies, ensuring that resources are allocated effectively and minimizing the risk of transmission. Ranking in this manner may allow tracking virus transmission in hospitals and may improve infection control efforts.

The integration of multiple methods of patient identification and authentication into the patient personal output apparatus may help ensure the accuracy and security of the system, and may allow early detection, tracking transmission, and finally prevention of sepsis.

### Sensors

Any of the apparatuses described herein may include one or more patient sensors, and/or may be used with one or more patient sensors. For example, any of these apparatuses may include noninvasive sensors (e.g., infrared, LADAR, etc.). In addition to utilizing patient data from EHRs and/or wearable devices, the patient personal output apparatuses described herein may be equipped with one or more sensors to monitor the physiological biomarkers of the patient. This may include a camera or LADAR system, Infrared Thermography sensor, or the like. These sensors, when directed to the patient, can monitor biomarkers such as blood pressure, skin color, movements, and other characteristics. Any of these apparatuses may include a microphone, which can analyze patient parameters, including, for example, patient sleep quality, e.g., by analyzing snore frequency. In some examples the apparatus may be equipped with a radio-frequency sensor to measure breathing patterns, providing a comprehensive picture of the patient's physiological status. Similarly the use of Infrared Thermography sensor allows for detection of changes in temperature caused by the patient's breathing and may convert them into respiratory rate readings, e.g., using sensor fusion techniques.

In general, these apparatuses may be configured to preserve the privacy of the patient. For example, in some cases the apparatus may be configured to provide privacy-preserving sensing. This may include the use of a privacy-preserving monocular camera depth estimators and other edge-computing methodologies to convert raw sound or visual recordings to abstract feature representations (via non-invertible transformations) for downstream interpretation by a trained neural network, e.g., using machine learning and deep learning processing. The use of such sensors, combined with trained networks (e.g., using deep learning algorithms) may provide a powerful tool for the early detection and prevention of decompensation (e.g., sepsis, kidney failure, heart failure, respiratory failure, etc.). The data collected by the sensors may be analyzed in real-time, allowing the apparatus to quickly identify potential decompensation events and provide a warning to caregivers. This innovative solution may offer a comprehensive and continuous monitoring system, improving the accuracy and effectiveness of detection.

The integration of these sensors and trained neural networks in the patient personal output apparatus may provide a powerful and comprehensive solution for the early detection and prevention e.g., of sepsis, and/or the continuous monitoring post-discharge, and improving patient outcomes and quality of life.

### Other Applications

As mentioned, the patient personal output apparatuses described herein may be used to detect, predict and/or warn a patient or caregiver about a variety of different decompensation risks, and are not limited to sepsis detection and prevention. For example, these apparatuses can be used as a bedside health coach to improve patients' sedation and agitation levels, delirium scales, and quality of sleep by monitoring medication doses, ambient light and noise level, and providing recommendations for improvements.

In a hospital setting, the patient's personal output apparatus can be used for monitoring volatile organic compounds (VOC) and particles that might transmit diseases, such as COVID-19. By continuously monitoring patients and detecting changes in physiological biomarkers, the device can provide an early warning of potential outbreaks, helping to prevent the spread of disease.

The patient personal output apparatuses described herein can be adapted for other health applications, such as monitoring and predicting chronic conditions, or monitoring and improving physical therapy outcomes. The patient personal output apparatuses described herein may also or alternatively be used for determining/detecting prescription adherence. For example, these apparatuses can receive information from electronic health records (EHRs) about the medications that a patient has been prescribed and track the intake of these medications over time. By continuously monitoring medication adherence, the patient personal output apparatus can help to improve patient outcomes by reducing the risk of medication-related adverse events and ensuring that patients receive the full benefits of their treatment. Additionally, by tracking medication intake over time, the apparatus may provide healthcare providers with valuable insights into patient behavior, helping them to make informed decisions about treatment.

Other potential applications may include, but are not limited to:

Eldercare: The patient personal output apparatus can be used to monitor the health of elderly patients and detect changes in their vital signs that may indicate a decline in health. Alternatively or additionally the apparatus may be integrated with wearable devices that track physical activity and send this information to the patient personal output apparatus, providing a more comprehensive view of elderly patients' health and wellness.

Sports Medicine: The patient personal output apparatus may be configured to monitor the health of athletes and detect changes in their vital signs that may indicate injury or overexertion. It can also be used to track physical activity and provide athletes with insights into their performance, helping them to improve their training and avoid injury.

Sleep Improvement: The patient personal output apparatus described herein may be used to analyze sleep patterns and to provide insights into the quality of sleep, helping patients to identify and address sleep issues. It can also be used to track the use of sleep-promoting technologies such as smart lights and white noise machines, and recommend adjustment of medications (given its access to patient's medical records and medication lists), thus helping patients to optimize their sleep environment.

Environment Monitoring: Any of the patient personal output apparatuses described herein may be used to monitor the environment, e.g., for particles that may transmit diseases, such as Covid-19. By continuously monitoring air quality, it can provide an early warning of potential health risks and help to reduce the spread of disease.

Clinical Research: The patient personal output apparatus described herein may be used to collect data on patients' vital signs and other health information, providing valuable insights into disease progression and the effectiveness of treatments. This information can be used to advance medical research and improve patient outcomes.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A patient personal output apparatus, the apparatus comprising:
an outer body configured to be positioned near a patient;
one or more outputs coupled to the outer body;
a control circuitry within the outer body, the control circuitry comprising:
a communications module configured to communicate with one or more of: the patient's electronic health record, or a monitoring device monitoring the patient to receive patient data;
a decompensation scoring module comprising a trained neural network configure to output a decompensation score based on the patient data; and
an output module configured to output a representation of the decompensation score from the one or more outputs.

2. The apparatus of claim 1, wherein the outer body is configured as an anthropomorphized animal shape.

3. The apparatus of claim 1, wherein the outer body is configured as a cartoon animal or mythical creature.

4. The apparatus of claim 1, wherein the one or more outputs comprises one or more LEDs.

5. The apparatus of claim 1, wherein the one or more outputs comprises a screen, display, projector, or speaker.

6. The apparatus of claim 1, wherein the control circuitry further comprises a memory for storing data from the patient's electronic health record, or a monitoring device monitoring the patient.

7. The apparatus of claim 1, wherein the control circuitry further comprises one or more non-contact sensors.

8. The apparatus of claim 7, wherein the one or more non-contact sensors comprises a camera, a LADAR subsystem, or infrared thermography subsystem.

9. The apparatus of claim 1, wherein the control circuitry further comprises a patient verification module configured to verify the identity of the patient.

10. The apparatus of claim 1, wherein the decompensation scoring module comprises a trained neural network configure to output a decompensation score representing risk of sepsis based on the patient data.

11. A patient personal output apparatus, the apparatus comprising:
an outer body configured to be positioned near a patient, wherein the outer body is configured as an anthropomorphized animal shape;
one or more outputs coupled to the outer body;
a control circuitry within the outer body, the control circuitry comprising:
a patient verification module configured to verify the identity of the patient;
a communications module configured to communicate with one or more of: the patient's electronic health record, or a monitoring device monitoring the patient to receive patient data;
a decompensation scoring module comprising a trained neural network configure to output a decompensation score representing risk of sepsis based on the patient data; and
an output module configured to output a representation of the decompensation score from the one or more outputs by modulating a light emitted by the one or more outputs.

12. A method, the method comprising:
receiving data specific to a patient from one or more of: a patient's electronic health record and/or a monitoring device associated with the patient, wherein the data is received by a control circuitry of a patient personal output apparatus;
determining, from a decompensation module of the patient personal output apparatus, a decompensation score based on the received data; and
outputting a visual indicator of the decompensation score from the patient personal output apparatus, wherein the patient personal output apparatus is positioned proximate to the patient.

13. The method of claim 12, further comprising associating the patient with the patient personal output apparatus.

14. The method of claim 12, further comprising: verifying the patient identity of a patient associated with a patient personal output apparatus.

15. The method of claim 12, wherein determining the decompensation score based on the received data comprises determining a risk of one or more of: sepsis, kidney failure, heart failure, or respiratory failure.

16. The method of claim 12, wherein determining the decompensation score based on the received data comprises determining a risk of sepsis.

17. The method of claim 12, wherein determining the decompensation score based on the received data comprises determining the decompensation score in real time in an ongoing manner.

18. The method of claim 12, wherein outputting the visual indicator of the decompensation score from the patient personal output apparatus comprises modulating a light emitted by the patient personal output apparatus while the patient personal output apparatus is positioned proximate to the patient.

19. The method of claim 12, wherein outputting the visual indicator of the decompensation score comprises changing the appearance of a region of the patient personal output apparatus corresponding to a region of the body related to the decompensation score.

20. A method, the method comprising:
associating the patient with a patient personal output apparatus that is positioned proximate to the patient, wherein the patient personal output apparatus comprises an anthropomorphized animal shape;
receiving data specific to a patient from one or more of: a patient's electronic health record and/or a monitoring device associated with the patient, wherein the data is received by a control circuitry of a patient personal output apparatus;
determining, from a decompensation module of the patient personal output apparatus, a decompensation score indicating a risk of sepsis in real time in an ongoing manner, and based on the received data; and
outputting a visual indicator of the decompensation score from the patient personal output apparatus by modulating a light emitted by the patient personal output apparatus while the patient personal output apparatus is positioned proximate to the patient.
